(19) United States
(12) Patent Application Publication
(10) Pub. No.: US 2020/0237627 A1
(43) Pub. Date: Jul. 30, 2020

(54) COSMETIC COMPOSITION FOR UV-SCREENING WITH COOLING EFFECT

(71) Applicant: COSMAX, INC., Hwaseong-si (KR)

(72) Inventors: Min Young YI, Yongin-si (KR); Sun Young KIM, Suwon-si (KR); Sung Ho KWON, Guro-gu (KR); Myeong Sam PARK, Gwangjin-gu (KR); Chun Ho PARK, Yongin-si (KR); Sung Yong KIM, Anyang-si (KR)

(73) Assignee: COSMAX, INC., Hwaseong-si (KR)

(21) Appl. No.: 16/743,279

(22) Filed: Jan. 15, 2020

(30) Foreign Application Priority Data
Jan. 25, 2019 (KR) 10-2019-0009942

(51) Int. Cl. A61K 8/04; A61K 8/37; A61K 8/69; A61Q 17/04; A61K 8/03; A61K 8/40; A61K 8/73; A61K 8/46

(52) U.S. Cl. CPC A61K 8/046; A61K 8/37; A61K 8/69; A61Q 17/04; A61K 2800/22; A61K 8/40; A61K 8/731; A61K 8/466; A61K 8/03

(57) ABSTRACT

The present disclosure relates to a cosmetic composition for UV screening in aerosol form, the composition including: an aerosol stock solution containing hydroxyethylcellulose and a UV-screening agent; and an aerosol propellant.

### CROSS-REFERENCE TO RELATED APPLICATION

This application claims the benefit of Korean Patent Application No. 10-2019-0009942, filed on Jan. 25, 2019, in the Korean Intellectual Property Office, the disclosure of which is incorporated herein in its entirety by reference.

### BACKGROUND

### 1. Field

The present invention relates to a cosmetic composition for UV screening in aerosol form, the composition including: an aerosol stock solution containing hydroxyethylcellulose and a UV-screening agent; and an aerosol propellant.

### 2. Description of Related Art

In general, products which provide a cooling sensation are mostly imparted with such a cool feeling effect by including volatile components such as ethanol, menthol, or derivatives thereof. Although such a method is effective for imparting a cooling sensation, the formulations of volatile components such as ethanol tend to be uniform without much difference, and the usage of menthol, which causes skin irritation, in UV-screening cosmetic compositions has been further limited. Therefore, there is a need for a novel formulation which can provide a new feeling along with a cooling sensation.

In aerosols which contain spraying gas, usage of such aerosols has been limited due to the gas and spraying pressure. It is possible to provide a mild cooling sensation using gas. However, no formulation has been able to provide a cooling sensation on its own without the gas-based methods. Therefore, there is a growing need for products with new aerosol formulations that are able to provide a cooling sensation.

Although previously studied cosmetic compositions, i.e., moisturizing cosmetic compositions with a cooling feeling which contain talc and butane, and cleansing cosmetic compositions in aerosol formulations can impart a cooling sensation to the skin, these cosmetic compositions have drawbacks such as the inability to form a bilayer phase structure due to the use of alkanolamide surfactants, the inclusion of expensive polymethyl methacrylate powder which reduces moisturization, and a lack of protection from UV.

In view of the above, the present inventors completed a cosmetic composition with a bilayered phase of gel and gas or a single liquid phase; and capable of imparting a new feeling as well as a cooling sensation in a different way from existing methods, and providing UV screening.

### PRIOR ART DOCUMENTS

### Patent Documents

(Patent document 1) KR 10-0602144

(Patent document 2) KR 10-2014-0134359

### SUMMARY

One aspect provides a cosmetic composition for UV screening, the composition including: an aerosol stock solution containing hydroxyethylcellulose and a UV-screening agent; and an aerosol propellant.

Additional aspects will be set forth in part in the description which follows and, in part, will be apparent from the description, or may be learned by practice of the presented embodiments of the disclosure.

One aspect provides a cosmetic composition for UV screening, the composition including: an aerosol stock solution containing hydroxyethylcellulose and a UV-screening agent; and an aerosol propellant.

An aerosol refers to a colloidal state in which solid or liquid particles are physically dispersed in gas. The cosmetic composition in aerosol form may refer to a spray agent in which an aerosol stock solution to be sprayed and a propellant are contained in a pressure-resistant container, such that the spray agent can uniformly spray cosmetics by the pressure of the propellant during spraying,

The aerosol propellant is not specifically limited; and may be any aerosol propellant available in cosmetic compositions. For example, the aerosol propellant may be at least one selected from the group consisting of nitrogen, liquefied petroleum gas (LPG), liquefied natural gas (LNG), dimethylether (DME), and tetrafluoropropene (HFO-1234ZE).

For example, an amount of the aerosol propellant may be about 10 wt % to about 70 wt %, and in some embodiments, about 20 wt % to about 70 wt %, and in some other embodiments, 30 wt % to about 70 et %, and in still other embodiments, about 40 wt % to about 60 wt %, with respect to a total weight of the cosmetic composition.

The aerosol stock solution, which is a material other than the aerosol propellant in the entire cosmetic composition, may refer to an effective ingredient which is sprayed to impart a specific effect. The aerosol stock solution may be in a liquid, powder, or cream state.

However, embodiments are not limited thereto

The aerosol stock solution may include a thickening agent and a UV-screening agent.

The term thickening agent can be interchangeably used with a viscosity increasing agent. The thickening agent may include any material capable of increasing the viscosity of the cosmetic composition. The thickening agent may be an inorganic thickening agent, an organic thickening agent, or a combination thereof. For example, the thickening agent may be at least one selected from the group consisting of bentonite, hectorite, PVP, lithium magnesium sodium silicate, sodium polyacrylate, sodium polyacrylate starch, corn starch, sodium stearate xanthan gum, ammonium acryloyldimethyltaurate/vp copolymer, acrylate/C10-30 alkylacrylate crosspolymer, carbomer, microcrystalline cellulose, cellulose gum, and hydroxyethylcellulose.

The thickening agent may be hydroxyethylcellulose. In one embodiment, the cosmetic composition for UV screening in aerosol formulation may impart a skin cooling sensation and a novel feeling by the inclusion of hydroxyethylcellulose. In particular, it was found that hydroxyethylcellulose may maximize the trapping function of the spray gas when the cosmetic composition in aerosol formulation is sprayed to control the properties of the composition upon spraying, thereby imparting, to the skin, a cooling effect and a new feeling, without polymethyl methacrylate. The cosmetic composition according to any of the embodiments was found to provide an improved cooling effect, without containing volatile components such as alcohols or menthol, which have been previously used to impart a cooling sensation.

For example, an amount of hydroxyethylcellulose may be about 0.1 wt % to about 2.0 wt %, and in some embodiments, about 0.3 wt % to about 1.8 wt %, and in some other embodiments, about 0.4% to about 1.6 wt %, and in still other embodiment, about 0.5 wt % to about 1.2 wt %, with respect to a total weight of the aerosol stock solution.

The UV-screening agent may be any material which is commonly used for the purpose of UV screening in cosmetic compositions. The UV-screening agent may be an organic UV-screening agent, an inorganic UV-screening agent, or a combination thereof. The organic UV-screening agent may be at least one selected from the group consisting of homosalate, ethylhexyl salicylate, phenylbenzimidazole sulfonic acid, octocrylene, ethylhexyl methoxycinnamate, butyl methoxydibenzoylmethane, bis-ethylhexyloxyphenol methoxyphenyl triazine, diethylamine hydroxybenzoyl hexyl benzoate, ethylhexyl triazone, isoamyl p-methoxycinnamate, disodium phenyl dibenzimidazole tetrasulfonate, and 4-methylbenzylidene camphor. The inorganic UV-screening agent may be at least one selected from the group consisting of titanium dioxide, zinc oxide, and zirconium oxide.

The cosmetic composition may not be a single-phase emulsion but may be a layered cosmetic composition in which at least two immiscible components form layers. The layered phase cosmetic composition may include: a water phase layer and at least one oil phase layer separated from the water phase layer. For example, the layer phase cosmetic composition may include two or three layers separated from one another. The order of the water phase layer and the oil phase layer may vary according to the composition of the cosmetic composition.

The oil phase layer may include oil, a component miscible with oil, or the organic UV-screening agent. The oil and the components miscible with oil may be any ingredients useful for the cosmetic composition. The oil phase layer may include, according to the layered phase structure of the composition, ester-based oils, for example, dipentaerythritol hexa C5-9 acid ester, C12-15 alkyloctanoate, myristyllactate, cetylethylhexanoate, neopentyl glycol dicaprate, cetyloctanoate, isopropyl myristate, octyldodecyl myristate, hexyl laurate, di(C12-C13) alkyl maleate, myristyl lactate, octyl dodecyl stearoyl stearate, diisotearyl maleate, pentaerythyl tetraethylhexanoate, or diglyceryl triisostearate; vegetable oils, for example, olive oil, grape seed oil, macadamia oil, jojoba oil, castor oil, sunflower seed oil, or lavender oil; silicon oils, for example, cyclopentasiloxane, cyclohexasiloxane, cycloheptasiloxane, cyclomethicone, cyclophenylmethicone, cyclotetrasiloxane, cyclotrisiloxanes dimethicone, dimethiconol, phenylmethicone, trifluoropropyl methicone, methyltrimethicone, or phenyltrimethicone; or a mixture of at least two thereof. For example, when a silicon oil having good miscibility with ester-based oils or vegetable oils is used, the cosmetic composition may have a two-layered phase structure including a water phase layer. For example, when dimethicone or dimethiconol is used, the cosmetic composition may be designed to have a three-layered phase including the water phase layer, each layer having a different specific density. A component other than the oil mixed in the oil phase layer may include cedrol, tocopherol, or retinol. Any component useful in the cosmetic composition may be included without limitation. The component mixed in the oil phase layer may be added in an amount which does not inhibit formation of multiple layers in the layered phase cosmetic composition according to an embodiment.

An amount of the oil may be about 1.0 wt % to about 50 wt %, and in some embodiments, about 10 wt % to about 50 wt %, and in some other embodiments, about 20 wt % to about 50 wt %, and in still other embodiments, about 30 wt % to 50 wt %, with respect to the total weight of the cosmetic composition. In one embodiment, when the cosmetic composition contains a high concentration of oil, the formulation may remain stable.

The cosmetic composition may be a single phase composition including a surfactant. The surfactant may be at least one selected from an anionic surfactant, a cationic surfactant, a non-ionic surfactant, an amphoteric surfactant, or a combination thereof. An amount of the surfactant may be about 0.1 wt % to 2.0 wt %, and in some embodiments, about 0.1 wt % to about 0.8 wt %, and in some other embodiments, 0.1 wt % to about 0.7 wt %, and in still other embodiments, about 0.1 wt % to about 0.5 wt %, with respect to a total weight of the cosmetic composition.

The cosmetic composition may include a coloring agent. The coloring agent may be applied to the skin or hair on the skin to develop a desired color, and may be included in the cosmetic composition without limitation as long as it does not inhibit an intended effect of the cosmetic composition. The coloring agent may be selected from a white coloring agent, a colored coloring agent, or a combination thereof. An amount of the coloring agent may be about 1.0 wt % to about 15 wt %, and in some embodiments, about 1.0 wt % to about 10 wt %, and in some other embodiments, about 1.0 wt % to about 8.0 wt %, and in still other embodiments, about 1.0 wt % to about 6.0 wt %, with respect to a total weight of the cosmetic composition.

The cosmetic composition may be a polyol or a derivative thereof for moisturizing and solvent purposes. The polyol may be any alcohol with multiple hydroxyl groups in one molecule, and any moisturizing agent known in the art may be included without limitation. For example, the polyol may be at least one selected from the group consisting of glycerin, diglycerin, 1,2-hexanediol, 1,3-butanediol, 1,4-butanediol, 1,5-pentanediol, sorbitol, mannitol, arabitol, xylitol, butylene glycol, propylene glycol, ethylene glycol, tetramethylene glycol, diethylene glycol, dipropylene glycol, hexylene glycol, pentylene glycol, pentaerythritol, polyethylene glycol, trimethylolpropane, and propanediol.

The cosmetic composition may further include, as necessary, a dispersant, an extender pigment, a coloring agent, an antioxidant, a preservative, a pH adjuster, a moisturizer, or a lubricant. The cosmetic composition for UV screening may additionally include a material which may supplement essential nutrients, for example, a natural fragrance, a cosmetic fragrance, or a plant extract. However, embodiments are not limited thereto, and the cosmetic composition may include any auxiliary agent.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a photograph showing the appearance of an aerosol formulation according to an embodiment after spraying;

FIG. 2 illustrates thermal infrared images of the skin before and after application of a cosmetic composition according to an embodiment of Example 7; and

FIG. 3 illustrates graphs of changes in skin surface temperature according to the application of a cosmetic composition according to an embodiment of Example 7.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### Examples 1 to 4 Preparation of Aerosol Stock Solution

To prepare aerosol stock solutions including hydroxyethylcellulose and a UV-screening agent, aerosol stock solutions of Examples 1 to 4 and Comparative Examples 1 and 2, including the components as shown in Table 1, were prepared according to the content ratios of ingredients shown in Table 2.

TABLE 1

| | | | | | | Comparative | Comparative |
|---|---|---|---|---|---|---|---|
| | Components | Example 1 | Example 2 | Example 3 | Example 4 | Example 1 | Example 2 |
| Aerosol stock | Purified water | to 100 | to 100 | to 100 | To 100 | to 100 | to 100 |
| solution | Polyol | 10.00 | 10.00 | 10.00 | 10 | 10.00 | 10.00 |
| | Hydroxyethylcellulose | 0.10 | 0.50 | 1.00 | 0.5 | 2.00 | — |
| | Oil phase | 40 | 40 | 40 | 30 | 40 | 40 |
| | Inorganic | — | — | — | 15 | — | — |
| | UV-screening agent | | | | | | |
| | Other additives | Appropriate | Appropriate | Appropriate | Appropriate | Appropriate | Appropriate |
| | | amount | amount | amount | amount | amount | amount |

In particular, polyol, hydroxyethylcellulose, and other additives were weighed and homogeneously mixed with purified water in a beaker using an emulsifying mixer at about 3,000 rpm at a temperature of about 75° C. At the same time, a UV-screening agent was weighed in a beaker and dissolved at a temperature of about 75° C. The dissolved UV-screening agent was added into the beaker containing the above mixture and homogeneously mixed at about 3,000 rpm for about 10 minutes. Then, the remaining additives were mixed therewith, cooled, and then degassed.

As a result, an aerosol stock solution containing purified water, polyol, hydroxyethylcellulose, and oil phase was prepared. The oil phase was prepared so as to contain oil, a component miscible with the oil phase layer, or an organic UV-screening agent. The aerosol stock solution of Example 4 further included an inorganic UV-screening agent.

In particular, the aerosol stock solutions of Examples 1 to 4 and Comparative Examples 1 and 2 further contained the ingredients as shown in Table 2.

TABLE 2

| | | | | | | Comparative | Comparative |
|---|---|---|---|---|---|---|---|
| | Ingredients | Example 1 | Example 2 | Example 3 | Example 4 | Example 1 | Example 2 |
| Aerosol | Purified water | 43.18 | 42.78 | 42.28 | 39.24 | 41.28 | 43.28 |
| stock | Butylene glycol (polyol) | 10.00 | 10.00 | 10.00 | 10.00 | 10.00 | 10.00 |
| solution | Hydroxyethylcellulose | 0.10 | 0.50 | 1.00 | 0.50 | 2.00 | — |
| | Dibutyl adipate (oil) | 3.00 | 3.00 | 3.00 | — | 3.00 | 3.00 |
| | Methyltrimethiocone (oil) | 5.00 | 5.00 | 5.00 | 3.00 | 5.00 | 5.00 |
| | C12-15alkylbenzoate (oil) | 5.00 | 5.00 | 5.00 | 4.00 | 5.00 | 5.00 |
| | Caprylylmethicone (oil) | — | — | — | 2.00 | — | — |
| | Cyclopentasiloxane (oil) | — | — | — | 11.00 | — | — |
| | bis-ethylhexyloxyphenol | 3.00 | 3.00 | 3.00 | — | 3.00 | 3.00 |
| | methoxyphenyl triazine | | | | | | |
| | (UV-screening agent) | | | | | | |
| | Ethylhexyl | 6.00 | 6.00 | 6.00 | 6.00 | 6.00 | 6.00 |
| | methoxycinnamate | | | | | | |
| | (UV-screening agent) | | | | | | |
| | Ethylhexyl salicylate | 5.00 | 5.00 | 5.00 | 4.00 | 5.00 | 5.00 |
| | (UV-screening agent) | | | | | | |
| | Butyl | 4.00 | 4.00 | 4.00 | — | 4.00 | 4.00 |
| | methoxydibenzoylmethane | | | | | | |
| | (UV-screening agent) | | | | | | |
| | Homosalate | 6.00 | 6.00 | 6.00 | — | 6.00 | 6.00 |
| | (UV-screening agent) | | | | | | |
| | Octocrylene | 3.00 | 3.00 | 3.00 | — | 3.00 | 3.00 |
| | (UV-screening agent) | | | | | | |
| | Phenylbenzimidazole | 3.50 | 3.50 | 3.50 | — | 3.50 | 3.50 |
| | sulfonic acid | | | | | | |
| | (UV-screening agent) | | | | | | |
| | Titanium dioxide | — | — | — | 15.00 | — | — |
| | (inorganic UV-screening | | | | | | |
| | agent) | | | | | | |
| | Disodium EDTA (extra | 0.02 | 0.02 | 0.02 | 0.02 | 0.02 | 0.02 |
| | additive) | | | | | | |
| | Polymethylsilsesquioxane | 2.00 | 2.00 | 2.00 | — | 2.00 | 2.00 |
| | (extra additive) | | | | | | |
| | Sodium chloride (extra | — | — | 1.00 | — | — | |
| | additive) | | | | | | |
| | Neutralizing agent (extra | 1.20 | 1.20 | 1.20 | — | 1.20 | 1.20 |
| | additive) | | | | | | |
| | Coloring agent (extra | — | — | — | 4.24 | — | — |
| | additive) | | | | | | |

### Examples 5 to 10. Preparation of Cosmetic Composition in Aerosol Formulation

To prepare cosmetic compositions for UV screening in aerosol formulation, the aerosol stock solutions of Examples 1 to 4 and Comparative Examples 1 and 2 were each used to prepare a two-layered phase liquid, which was then filled into a pressure-resistant container in an optimal ratio to thereby prepare the aerosol cosmetic composition.

In particular, the cosmetic compositions of Examples 5 to 10 and Comparative Examples 3 to 6 were prepared according to the amounts of the aerosol stock solution and the types and amounts of an aerosol propellant as shown in Table 3.

TABLE 3

| | | Example | Example | Example | Example | Example | Example | Comparative | Comparative | Comparative | Comparative |
|---|---|---|---|---|---|---|---|---|---|---|---|
| Ingredient | Example | 5 | 6 | 7 | 8 | 9 | 10 | Example 3 | Example 4 | Example 5 | Example 6 |
| Aerosol | Example 1 | 80.00 | | | | | | — | 20.00 | — | — |
| stock | Example 2 | | 80.00 | 60.00 | | | | 95.00 | — | | |
| solution | Example 3 | | | | 70.00 | 40.00 | | | | | |
| content | Example 4 | | | | | | 60.00 | | | | |
| | Comparative | | | | | | | | | | 70.00 |
| | Example 1 | | | | | | | | | | |
| | Comparative | | | | | | | | | 60.00 | |
| | Example 2 | | | | | | | | | | |
| Propellant | Liquefied | 20.00 | | 40.00 | 30.00 | | 40.00 | 5.00 | 80.00 | | 30.00 |
| content | petroleum gas | | | | | | | | | | |
| | Dimethyl ether | | 20.00 | | | 60.00 | | | | 40.00 | |

### Experimental Example 1. Evaluation of Cosmetic Composition in Terms of Aerosol Formulation Formation, Cooling Sensation, and Feeling

Visual evaluation and evaluation of feeling were performed on the cosmetic compositions of Examples 5 to 10 and Comparative Examples 3 to 6.

In particular, the formation of the aerosol formulation was determined through visual evaluation. The cooling effect was evaluated through an in-house efficacy test by measuring the temperature of the skin over time after application of each cosmetic composition. In the evaluation of feeling; a panel of nine individuals aged between 25 and 35 were asked to evaluate the novelty of feeling on a 10-point scale (10 being the highest).

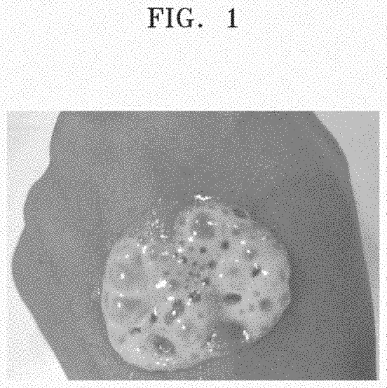

The results are shown in Table 4. The appearance of the cosmetic formulation of Example 7 after spraying is shown in FIG. 1.

TABLE 4

| | | Example | Example | Example | Example | Example | Example | Comparative | Comparative | Comparative | Comparative |
|---|---|---|---|---|---|---|---|---|---|---|---|
| | Item | 5 | 6 | 7 | 8 | 9 | 10 | Example 3 | Example 4 | Example 5 | Example 6 |
| Evaluation | Formulation | O | O | O | C | O | C | X | O | X | X |
| results | formation | | | | | | | | | | |
| | Cooling | Yes | Yes | Yes | Yes | Yes | Yes | Slight | Yes | Slight | Slight |
| | sensation | | | | | | | | | | |
| | Novelty of | 7.89 | 8.00 | 9.22 | 9.00 | 8.56 | 8.67 | — | 5.11 | — | — |
| | feeling | | | | | | | | | | |

As shown in Table 4, the cosmetic composition of Comparative Example 3 having an aerosol propellant content of less than 10 wt % failed to form an aerosol formulation, and the cosmetic composition of Comparative Example 4 having an aerosol propellant content of greater than 74 wt % blew away easily such that usability was poor. The cosmetic composition of Comparative Example 5 including no hydroxyethylcellulose failed to form an aerosol formulation, even though it contained an appropriate amount of aerosol propellant. The cosmetic composition of Comparative Example 6 including excess hydroxyethylcellulose also failed to form an aerosol formulation.

However, the cosmetic composition of Example 7 was found to form an aerosol formulation, due to the inclusion of hydroxyethylcellulose in the aerosol stock solution, even though it contained the aerosol propellant in the same ratio as in the cosmetic composition of Comparative Example 5, and was also found to exhibit a strong cooling sensation and a highly novel feeling.

That is, even if the aerosol stock solution contains an oil phase, specifically, a high content of oil, it was found to be possible to form an aerosol formulation and impart a cooling sensation and an improved feeling when the aerosol stock solution includes hydroxyethylcellulose.

The cosmetic compositions of Examples 5 to 10 were also found to be sprayed in a slush state as shown in FIG. 1.

### Experimental Example 2 Sensory Test of Cosmetic Composition

A sensory test was performed on the cosmetic compositions in aerosol formulation according to Examples 5 to 10 and Comparative Example 3. In particular, in the sensory test, nine panelists aged between 25 and 35 were asked to evaluate novelty of feeling on a 10-point scale (10 being the highest).

The results are shown in Table 5.

TABLE 5

| | | | | | | | Comparative |
|---|---|---|---|---|---|---|---|
| | Example 5 | Example 6 | Example 7 | Example 8 | Example 9 | Example 10 | Example 3 |
| Evaluator A | 8 | 8 | 9 | 9 | 9 | 9 | 6 |
| Evaluator B | 8 | 8 | 10 | 9 | 9 | 9 | 6 |
| Evaluator C | 7 | 8 | 9 | 7 | 8 | 8 | 4 |
| Evaluator D | 7 | 9 | 9 | 9 | 9 | 9 | 6 |
| Evaluator E | 9 | 8 | 10 | 10 | 9 | 10 | 6 |
| Evaluator F | 8 | 8 | 9 | 9 | 8 | 8 | 5 |
| Evaluator G | 9 | 7 | 9 | 10 | 8 | 8 | 5 |
| Evaluator H | 8 | 9 | 10 | 10 | 9 | 9 | 6 |
| Evaluator I | 7 | 7 | 8 | 8 | 8 | 8 | 2 |

As represented in Table 5, most of the evaluators evaluated, though with slight individual differences, that improved feeling was felt with the cosmetic compositions of Examples 5 to 10 prepared using one of the aerosol stock solutions of Examples 1 to 4. In particular, the cosmetic compositions of Examples 7 to 10 containing about 40 wt % to about 60 wt % of the aerosol propellant were scored high points in the evaluation test. However, the cosmetic composition of Comparative Example 3 using the aerosol stock solution of Example 2 but having an aerosol propellant content of 5 wt % failed to form a desired aerosol formulation, and was evaluated not to exhibit improved feeling.

### Experimental Example 3 Confirmation of Skin Surface Temperature Reduction Effect According to Application of Cosmetic Composition

The cosmetic composition of Example 7 was applied to the skin to confirm a skin surface temperature reduction effect. In particular, a non-application area and application area for the cosmetic composition were set on the arms of a panel of five individuals. Thermal infrared images were captured before and after the application of the cosmetic composition and compared. In the thermal infrared images, purple, red, and black areas are observed, wherein, the closer to purple, the lower the temperature.

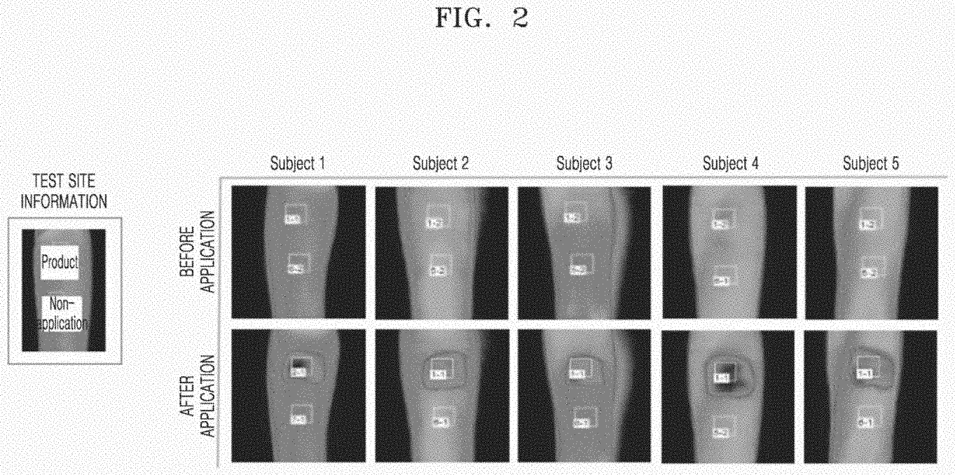

As a result, as shown in FIG. 2, a visual difference between the application area and the non-application area was not observed before the application of the cosmetic composition. However, after the application of the cosmetic composition, a significant effect of skin temperature reduction in the application area was even visually confirmed.

The average skin surface temperature before and after the application of the cosmetic composition was also measured. The average skin surface temperature before the application of the cosmetic composition was 26.3° C., and a surface temperature reduction rate was also identified based on this temperature.

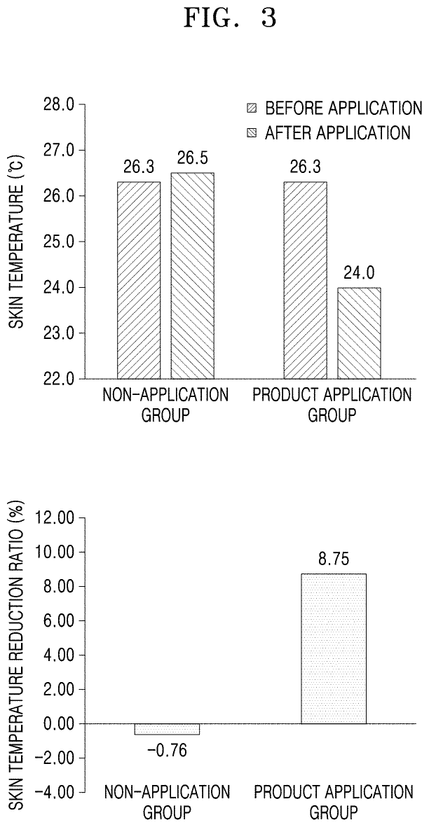

As a result, as shown in FIG. 3, when the cosmetic composition was not applied, the rate of change in skin surface temperature was insignificant. Rather, the skin surface temperature increased due to environmental impacts. Unlike this, when the cosmetic composition of Example 7 was applied, the average skin surface temperature was found to be reduced from 26.3° C. to 24.0° C., with a skin surface temperature rate of about 8.7%.

From the above results, it was confirmed that the cosmetic composition for UV screening may impart improved cooling sensation, due to the inclusion of hydroxyethylcellulose, better than a cooling sensation merely by the gas of an aerosol formulation

It was also found that a desired aerosol formulation is not formed when the aerosol propellant content is less than 10 wt %, there are concerns about frostbite and blowing away of the contents resulting in poor feeling, due to too strong spraying pressure, when the aerosol propellant content is greater than 70 wt %, and improved cooling sensation and feeling result in when the aerosol propellant content is about 10 wt % to about 70 wt %.

As described above, according to the one or more embodiments, a cosmetic composition for UV screening in aerosol formulation may impart, by using an aerosol propellant having a low melting point and hydroxyethylcellulose, a cooling sensation and a novel feeling when being sprayed.

It should be understood that embodiments described herein should be considered in a descriptive sense only and not for purposes of limitation, Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A cosmetic composition for UV screening, the composition comprising:
an aerosol stock solution including hydroxyethylcellulose and a UV-screening agent; and
an aerosol propellant.

2. The cosmetic composition of claim 1, wherein an amount of the aerosol propellant is about 10 wt % to about 70 wt % with respect to a total weight of the corn position.

3. The cosmetic composition of claim 1, wherein an amount of the hydroxyethylcellulose is about 0.1 wt % to about 1.8 wt % with respect to a total weight of the aerosol stock solution.

4. The cosmetic composition of claim 1, wherein the aerosol propellant is at least one selected from the group consisting of nitrogen, liquefied petroleum gas (LPG), liquefied natural gas (LNG), dimethylether (DME), and tetrafluoropropene (HFO-1234ZE).

5. The cosmetic composition of claim 1, wherein the UV-screening agent is at least one selected from the group consisting of homosalate, ethylhexyl salicylate, phenylbenzimidazole sulfonic acid, octocrylene, ethylhexyl methoxycinnamate, butyl methoxydibenzoylmethane, bis-ethylhexyloxyphenol methoxyphenyl triazine, diethylamine hydroxybenzoyl hexyl benzoate, ethylhexyl triazone, isoamyl p-methoxycinnamate, disodium phenyl dibenzimidazole tetrasulfonate, and 4-methylbenzylidene camphor.

6. The cosmetic composition of claim 1, wherein the composition comprises a water phase layer including the aerosol stock solution and the aerosol propellant, and an oil phase layer separated from the water phase layer, and
the composition comprises about 1.0 wt % to about 50 wt % of the oil phase layer with respect to a total weight of the composition.

7. The cosmetic composition of claim 1, wherein, upon spraying, the composition is sprayed in a slush state.
